(19) United States
(12) Patent Application Publication
(10) Pub. No.: US 2023/0293184 A1
(43) Pub. Date: Sep. 21, 2023

(54) INTERFERENCE FEATURE FOR INHIBITING PREMATURE EMBOLIC IMPLANT DETACHMENT

(71) Applicant: DePuy Synthes Products, Inc., Raynham, MA (US)

(72) Inventors: Daniel SOLAUN, Miami, FL (US); Lacey GOROCHOW, Raynham, MA (US); Joshua FARIA, Campbell, CA (US)

(73) Assignee: DePuy Synthes Products, Inc., Raynham, MA (US)

(21) Appl. No.: 17/694,777

(22) Filed: Mar. 15, 2022

(51) Int. Cl. A61B 17/12

(52) U.S. Cl. CPC A61B 17/1214; A61B 17/12113; A61B 2017/12054

(57) ABSTRACT

Various systems and methods of delivering an implant are disclosed. A detachment system can include an outer tubular body defining a longitudinal axis that includes a first lumen, and an inner tubular body that includes a second lumen extending therethrough with the inner tubular body disposed within the first lumen. A pull wire can be at least partially within the second lumen and at least partially within the first lumen, and configured to translate in a proximal direction in relation to the outer tubular body to release the implant. A radial lumen can extend through the outer tubular body and inner tubular body orthogonal to the longitudinal axis. An interference feature can at least partially extend through the radial lumen to inhibit premature detachment of the implant. The interference feature can fracture in response to proximal translation of the inner tubular body to facilitate release of the implant.

### FIELD OF INVENTION

The present invention relate to aneurysm treatment devices and more particularly, to improved delivery systems for embolic implants that prevent premature implant deployment.

### BACKGROUND

Numerous intravascular implant devices are known in the field. Many are deployed mechanically, via systems that combine one or more catheters and wires for delivery. Examples of implants that can be delivered mechanically include embolic elements, stents, grafts, drug delivery implants, flow diverters, filters, stimulation leads, sensing leads, or other implantable structures delivered through a microcatheter. Some obstetric and gastrointestinal implants may also be implanted via similar systems that combine one or more catheters and wires. Devices that may be released or deployed by mechanical means vary greatly in design but can employ a similar delivery catheter and wire system. Many such catheter-based delivery systems include a wire for retention of the implant in the catheter until the time for release of the device. These systems are then actuated by retracting or pulling the wire relative to the catheter. Such a wire is referred to herein as a “pull wire”.

One issue with current catheter-based delivery systems is premature detachment of the implantable device. Premature detachment occurs when the implant is detached from the delivery system before reaching the treatment site. This may occur due to the tortuosity experienced by the delivery system as it passes through the vasculature of the patient, which can cause an increase in friction between the “pull wire” and the delivery system causing the pull wire to move proximally while the delivery system is moving distally.

Accordingly, there is a need for an improved implant delivery system that prevents premature detachment of the implant as it is delivered through tortuous vasculature. This disclosure is directed to this and other considerations.

### SUMMARY

Various systems and methods of delivering an implant are disclosed. A detachment system can include an outer tubular body defining a longitudinal axis that includes a first lumen, and an inner tubular body that includes a second lumen extending therethrough with the inner tubular body disposed within the first lumen. A pull wire can be at least partially within the second lumen and at least partially within the first lumen, and configured to translate in a proximal direction in relation to the outer tubular body to release the implant. A radial lumen can extend through the outer tubular body and inner tubular body orthogonal to the longitudinal axis. An interference feature can at least partially extend through the radial lumen to inhibit premature detachment of the implant. The interference feature can fracture in response to proximal translation of the inner tubular body to facilitate release of the implant.

In one aspect, a detachment system for delivering an implantable medical device to a target location of a body vessel is disclosed. The detachment system can include an outer tubular body that includes a first lumen that extends therethrough along a longitudinal axis. The detachment system can include an inner tubular body that includes a second lumen extending therethrough along the longitudinal axis. The inner tubular body can be disposed within the first lumen of the outer tubular body. The detachment system can include a pull wire that is disposed at least partially within the second lumen of the inner tubular body and at least partially within the first lumen of the outer tubular body. The pull wire can be configured to translate in a proximal direction in relation to the outer tubular body to release the implantable medical device from a distal end of the detachment system. The detachment system can include a radial lumen that extends through the outer tubular body and the inner tubular body orthogonal to the longitudinal axis. The detachment system can include an interference feature at least partially extending through the radial lumen. The interference feature can be effective to inhibit premature detachment of the implantable medical device as the implantable medical device is delivered by the detachment system to the target location of the body vessel. The interference feature can be configured to fracture in response to proximal translation of the inner tubular body to thereby facilitate release of the implantable medical device from the detachment system.

In some embodiments, the interference feature can include a monofilament suture.

In some embodiments, the interference feature is constructed of polypropylene 8-0.

In some embodiments, the interference feature is secured to an outer wall of the outer tubular body by a method selected from welding the interference feature to the outer wall, melting the interference feature to the outer wall, knotting the interference feature to the outer wall, and gluing the interference feature to the outer wall.

In some embodiments, the interference feature prevents proximal translation of the inner tubular body with respect to the outer tubular body.

In some embodiments, the proximal end of the pull wire can be attached to a proximal end of the inner tubular body. The pull wire can be configured to translate with the inner tubular body as a single unit in relation to the outer tubular body.

In some embodiments, the radial lumen can include an outer radial lumen and an inner radial lumen. The outer radial lumen can be in alignment with the inner radial lumen as the implantable medical device is delivered by the detachment system to the target location of the body vessel. The inner radial lumen can be configured to move out of alignment with respect to the outer radial lumen to thereby fracture the interference feature and facilitate release of the implantable medical device from the detachment system.

In some embodiments, the interference feature can extend through an entire length of the radial lumen and the interference feature can be secured to an outer wall of the outer tubular body at each end of the radial lumen.

In some embodiments, the interference feature is configured to fracture in response to a force between approximately 50 gram-force and approximately 100 gram-force.

In some embodiments, the detachment system can further include a loop wire that includes a loop opening that is positioned approximate a distal end of the outer tubular body. The loop wire and the pull wire can be positioned to secure the implantable medical device to the delivery system.

In some embodiments, the outer tubular body can further include a compressed distal portion that is held in compression by tension in the loop wire and the can be configured to provide a force distally to the implantable medical device upon release of the implantable medical device from the distal end of the detachment system.

In another aspect a method is disclosed. The method can include providing an outer tubular body that includes a first lumen extending therethrough along a longitudinal axis. The method can include providing an inner tubular body including a second lumen extending therethrough along the longitudinal axis. The method can include positioning the inner tubular body within the first lumen of the outer tubular body. The method can include forming a radial lumen that can extend radially through the outer tubular body and the inner tubular body orthogonal to the longitudinal axis. A pull wire can be extended through the second lumen of the inner tubular body and into the first lumen of the tubular body. A proximal end of the pull wire can be secured to a proximal end of the inner tubular body. An interference feature can be extended at least partially through the radial lumen such that the interference feature extends through an outer wall of the outer tubular body and an inner wall of the inner tubular body. The method can include securing an implantable medical device to the outer tubular body such that proximal translation of the pull wire can release the implantable medical device from the outer tubular body. The method can include preventing, with the interference feature, release of the implantable medical device while the implantable medical device is delivered through vasculature to a treatment site. The method can include translating the inner tubular body proximally to thereby fracture the interference feature and facilitate release of the implantable medical device from the detachment system.

In some embodiments, the radial lumen can include an outer radial lumen and an inner radial lumen. The outer radial lumen can be in alignment with the inner radial lumen as the implantable medical device is delivered by the detachment system to the target location of the body vessel. The inner radial lumen can be configured to move out of alignment with respect to the outer radial lumen to thereby fracture the interference feature and facilitate release of the implantable medical device from the detachment system.

In some embodiments, the method can include affixing a loop wire to the outer tubular body and positioning a loop opening in the loop wire approximate a distal end of the outer tubular body and through a locking portion of the implantable medical device.

In some embodiments, the interference feature can be configured to fracture in response to a force between approximately 50 gram-force and approximately 100 gram-force.

In some embodiments, the method can include preventing proximal translation of the inner tubular body with respect to the outer tubular body while the implantable medical device is delivered through vasculature to the treatment site.

In some embodiments, the method can include securing the interference feature to the outer wall of the outer tubular body using a method selected from welding the interference feature to the outer wall, melting the interference feature to the outer wall, knotting the interference feature to the outer wall, and gluing the interference feature to the outer wall.

In some embodiments, extending the interference feature through the radial lumen can include extending a body of the interference feature through an entire length of the radial lumen and attaching first and second ends of the interference feature to the outer wall at respective ends of the radial lumen.

In some embodiments, the body of the interference feature can be a monofilament suture.

In some embodiments, the pull wire can translate proximally with the inner tubular body as a single unit.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIGS. 1A-1E are illustrations of an exemplary detachment system and implant, according to aspects of the present invention.

FIGS. 2A-2E are illustrations of another exemplary detachment system and implant, according to aspects of the present invention.

FIG. 3 is an illustration of a delivery system navigating a body lumen according to aspects of the present invention.

FIG. 4 is an illustration of embolic coils being positioned within an aneurysm according to aspects of the present invention.

FIGS. 5A-5D illustrate a sequence of steps for releasing an embolic implant from the delivery member, according to aspects of the present invention.

FIG. 6 is a flowchart of an example method of using the detachment system, according to aspects of the present invention.

### DETAILED DESCRIPTION

The following description of certain examples of the invention should not be used to limit the scope of the present invention. The drawings, which are not necessarily to scale, depict selected embodiments and are not intended to limit the scope of the invention. The detailed description illustrates by way of example, not by way of limitation, the principles of the invention. Other examples, features, aspects, embodiments, and advantages of the invention will become apparent to those skilled in the pertinent art from the following description, which is by way of illustration, one of the best modes contemplated for carrying out the invention. As will be realized, the invention is capable of other different or equivalent aspects, all without departing from the invention. Accordingly, the drawings and descriptions should be regarded as illustrative in nature and not restrictive.

Any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those skilled in the pertinent art in view of the teachings herein. Such modifications and variations are intended to be included within the scope of the claims.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment.

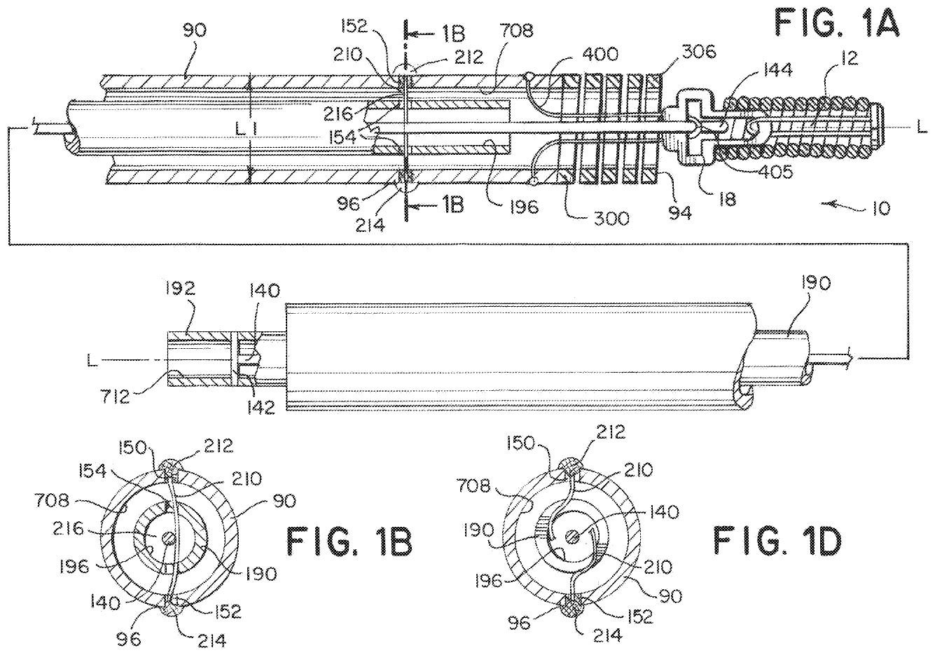

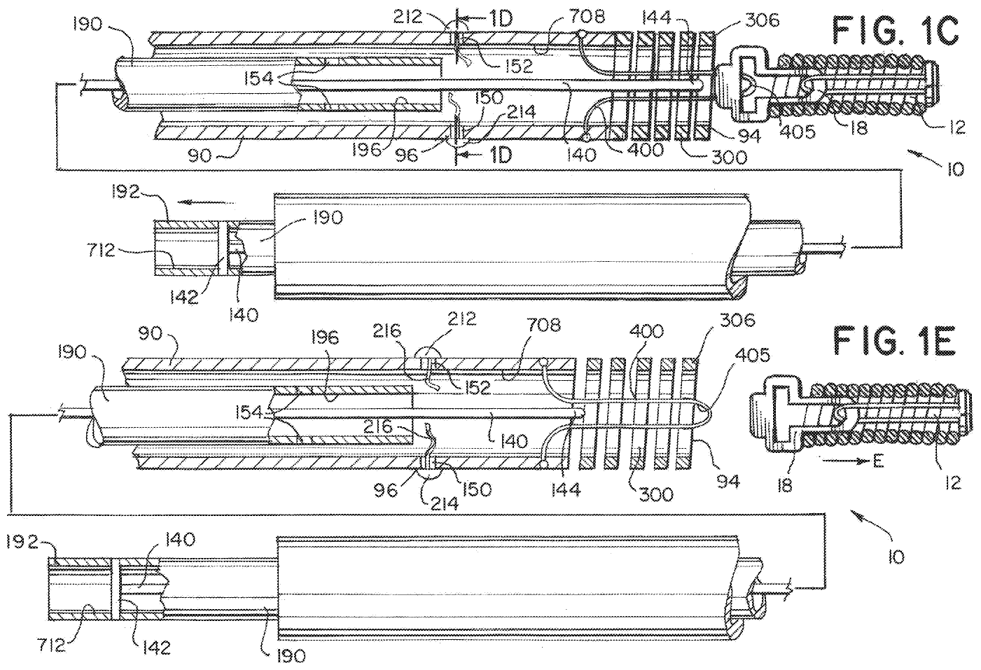

Turning to the figures, FIGS. 1A-1E shows an example detachment system 10 for delivering an implantable medical device 12 to a target location of a body vessel. More specifically, FIGS. 1A-1E show a sequence of steps for detaching an implantable medical device 12 from the detachment system 10. The example detachment system 10 can include an outer tubular body 90 having a first lumen extending therethrough. The outer tubular body 90 can define a longitudinal axis L-L that extends along a length of the detachment system 10. Disposed within lumen 708 may be an inner tubular body 190. The inner tubular body 190 can include a second lumen 712 that extends therethrough along the longitudinal axis L-L. Disposed at least partially within the second lumen 712 of the inner tubular body 190 and at least partially within the first lumen 708 of the outer tubular body 90, a pull wire 140 can extend along the longitudinal axis from a proximal end 192 of inner tubular body 190 to a distal end 94 of outer tubular body 90. The pull wire can include a proximal end 142 that is attached to the proximal end 192 of the inner tubular body 190. Accordingly, when inner tubular body 190 is translated proximally with respect to outer tubular body 90, the pull wire 140 can translate proximally with inner tubular body 190 as a single unit. Proximal end 142 of pull wire 140 can be attached to proximal end 192 of inner tubular body 190 in any way as would be understood by a person having skill in pertinent art. For example, proximal end 142 of pull wire 140 can be welded or glued to proximal end 192 of inner tubular body 190, although other methods of attachment are not precluded. Pull wire 140 can be constructed out of any suitable material, for example, pull wire 140 can be constructed of stainless steel or memory shape material, such as nitinol. According to some embodiments, pull wire 140 can additionally be coated with polytetrafluoroethylene (PTFE).

Respective proximal ends of a loop wire 400 can be attached to the outer tubular body 90 of the detachment system 10. Disposed at a distal end of loop wire 400 can be a loop opening 405. Loop opening 405 can be passed through a locking portion 18 (e.g., aperture) of an implantable medical device 12. After loop opening 405 is positioned through locking portion 18, a distal end 144 of pull wire 140 can be positioned through the loop opening 405, thereby securing the implantable medical device 12 to the detachment system 10. Just distal of where respective proximal ends of loop wire 400 are attached to the outer tubular body 90, the outer tubular body 90 can include a compressed distal portion 300. The compressed distal portion 300 can be formed from spiral-cuts 306 formed in the outer tubular body 90, which can be formed by a laser cutting operation. Additionally, or alternatively, the compressible distal portion 300 can be formed of a wound wire, spiral ribbon, or other arrangement allowing axial adjustment according to the present invention. Preferably, compressible distal portion 300 is in the elongated condition at rest and automatically or resiliently returns to the elongated condition from a compressed condition, unless otherwise constrained. In some embodiments, the implantable medical device 12 can be an embolic coil.

Disposed orthogonally to the longitudinal axis L-L can be a radial lumen 150 that extends through the outer tubular body 90 and the inner tubular body 190. For example, the radial lumen can be formed by creating apertures within an outer tubular body wall 96 of outer tubular body 90 and inner tubular body 190 such that radial lumen 150 runs through respective lumens 708, 712. Radial lumen 150 can include an outer radial lumen 152 and an inner radial lumen 154, which are in alignment and collectively form radial lumen 150 while the detachment system 10 is delivered to a treatment site, as shown in FIG. 1A. The radial lumen 150 can have a length L1 that is approximately equal to the diameter of detachment system 10 as measured radially (e.g., orthogonal to longitudinal axis L-L) through outer tubular body 90. An interference feature 210a can be positioned through radial lumen 150. As shown in FIGS. 1A-1E, interference feature 210a can include a first end 212, a second end 214, and an interference feature body 216. The interference feature first end 212 can be disposed against an outer tubular body wall 96 on a first side of the radial lumen 150. Opposite the first side of radial lumen 150, the interference feature second end 214 can be disposed against outer tubular body wall 96 on a second side of the radial lumen 150. The interference feature body 216 can extend from the first end 212, through radial lumen 150, and terminate at the second end 214. The interference feature 210a can be constructed of any suitable material as would be understood by a person having skill in the pertinent art. According to some embodiments, the interference feature 210a can be a polymeric mono filament structure, such as a suture. In some embodiments, the interference feature 210a can be constructed of a polymer such as polypropylene 8-0. The interference feature 210a can have advantageous physical properties, such as having a predictable break load. In some embodiments, interference feature 210a can be attached to the outer wall 96 of the outer tubular body 90 by welding respective ends 212, 214 of interference feature 210a to respective ends of the outer wall 96. In some embodiments, interference feature 210a can be attached to the outer wall 96 by melting respective ends 212, 214 of interference feature 210a to respective ends of the outer wall 96. In some embodiments, the interference feature 210a can be attached to the outer wall 96 by knotting the interference feature 210a to the outer wall 96. In yet other embodiments, the interference feature 210a can be attached to the outer wall 96 by gluing respective ends 212, 214 of interference feature 210a to respective ends of the outer wall 96.

Now turning to FIG. 1A, the detachment system 10 is shown as it is being delivered to a treatment site through tortuous vasculature. As the detachment system 10 is passes through the vasculature, the interference feature 210a prevents the proximal translation of inner tubular body 190 with respect to outer tubular body 90. Accordingly, the interference feature 150 can be effective to prevent premature detachment of the implantable medical device 12 from detachment system 10 as it prevents the inner tubular body 190 and pull wire 140 from being translated proximally with respect to outer tubular body 90. FIG. 1B shows a cross-sectional view of detachment system 10 as it is being delivered to a treatment site through tortuous vasculature. As shown, interference feature 210a can extend through radial lumen 150 to prevent proximal translation of inner tubular body 190 with respect to outer tubular body 90. According to some embodiments, radial lumen 150 can be slightly offset from a center axis of delivery system 10 in order to sufficiently space interference feature 210a from pull wire 140 such that interference feature 210a does not touch or rub against pull wire 140.

FIG. 1C shows the detachment system 10 at the moment that a sufficient force in a proximal direction is applied to inner tubular body 190, thereby causing interference feature 210a to fracture and facilitating the distal end 144 of pull wire 140 to be translated proximally. As shown in FIG. 1C, distal end 144 of pull wire 140 is translated proximally and the distal end 144 of pull wire 140 exits loop opening 405, thereby facilitating release of implantable medical device 12 from detachment system 10. As can be seen in FIG. 1C, an outer radial lumen 152 is now out of alignment with respect to inner radial lumen 154. According to some embodiments, the interference feature 210a is configured to fracture in response to a force between approximately 50 gram-force and 100 gram-force. FIG. 1D shows a cross-sectional view of detachment system 10 as the moment that a sufficient force in a proximal direction is applied to inner tubular body 190, thereby causing interference feature 210a to fracture and facilitating the distal end 144 of pull wire 140 to be translated proximally. As shown, interference feature 210a can fracture and no longer prevent inner tubular body 190 from translating proximally with respect to outer tubular body 90.

FIG. 1E shows the detachment system 10 with detached implantable medical device 12. Following proximal translation of pull wire 140, the compressed distal section 300 can elongate into its uncompressed state and impart an elastic force E against the implantable medical device 12, facilitating a clean release from the detachment system 10 and effective deployment to the treatment site.

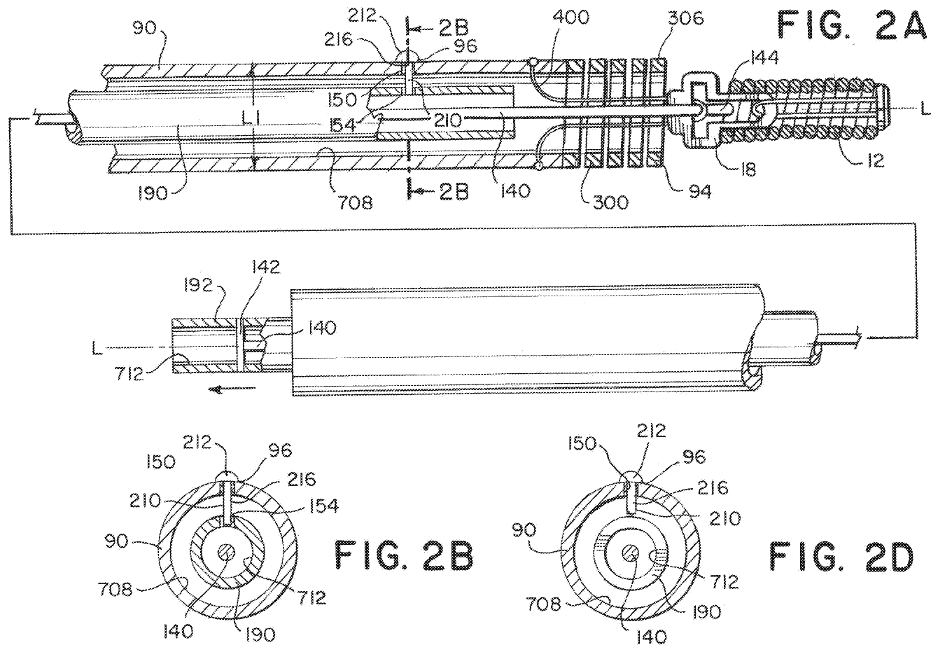

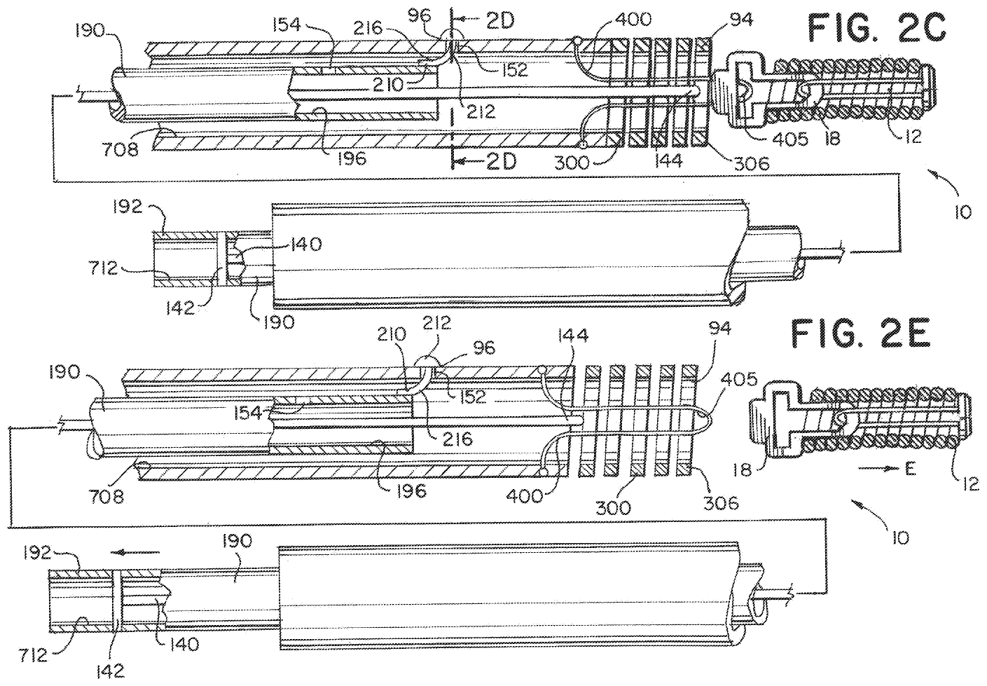

FIGS. 2A-2E are illustrations of another exemplary detachment system and implant, according to aspects of the present invention. As shown in FIGS. 2A-2E, the detachment system may have an interference feature 210b that only partially extends through radial lumen 150. The interference feature 210b can be constructed of any suitable material as would be understood by a person having skill in the pertinent art. According to some embodiments, the interference feature 210b can be a polymeric mono filament structure, such as a suture. In some embodiments, the interference feature 210b can be constructed of a polymer such as polypropylene 8-0. The interference feature 210b can have advantageous physical properties, such as having a predictable break load. In some embodiments, interference feature 210b can be attached to the outer wall 96 of the outer tubular body 90 by welding end 212 of interference feature 210b to the outer wall 96. In some embodiments, interference feature 210b can be attached to the outer wall 96 by melting end 212 of interference feature 210b to the outer wall 96. In some embodiments, the interference feature 210b can be attached to the outer wall 96 by knotting the interference feature 210b to the outer wall 96. In yet other embodiments, the interference feature 210b can be attached to the outer wall 96 by gluing end 212 of interference feature 210b to the outer wall 96.

As shown in FIG. 2A, the interference feature 210b can extend partially through radial lumen 150 while passing through outer tubular body wall 96 and inner tubular body wall 196 on one end of outer tubular body 90 and an inner tubular body 190, respectively. FIG. 2B shows a cross-sectional view of detachment system 10 as it is being delivered to a treatment site through tortuous vasculature. As shown, interference feature 210b can extend through outer radial lumen 152 and inner radial lumen 154 to prevent proximal translation of inner tubular body 190 with respect to outer tubular body 90.

FIG. 2C shows the detachment system 10 at the moment that a sufficient force is applied in a proximal direction to inner tubular body 190, thereby causing the detachment feature 210b to break or bend such that detachment feature 210b no longer interferes with inner tubular body 190. As can be seen in FIG. 2C, once the inner tubular body 190 is translated proximally, inner radial lumen 154 moves out of alignment with respect to outer radial lumen 152. According to some embodiments, the interference feature 210b is configured to fracture or bend in response to a force between approximately 50 gram-force and 100 gram-force. FIG. 2D shows a cross-sectional view of detachment system 10 as the moment that a sufficient force in a proximal direction is applied to inner tubular body 190, thereby causing interference feature 210b to fracture or bend and facilitate the distal end 144 of pull wire 140 to be translated proximally. As shown, interference feature 210b can fracture or bend such that interference feature 210b no longer extends into inner radial lumen 154 and thus no longer prevents inner tubular body 190 from translating proximally with respect to outer tubular body 90.

FIG. 2E shows the detachment system 10 with detached implantable medical device 12. Following proximal translation of pull wire 140, the compressed distal section 300 can elongate into its uncompressed state and impart an elastic force E against the implantable medical device 12, facilitating a clean release from the detachment system 10 and effective deployment to the treatment site.

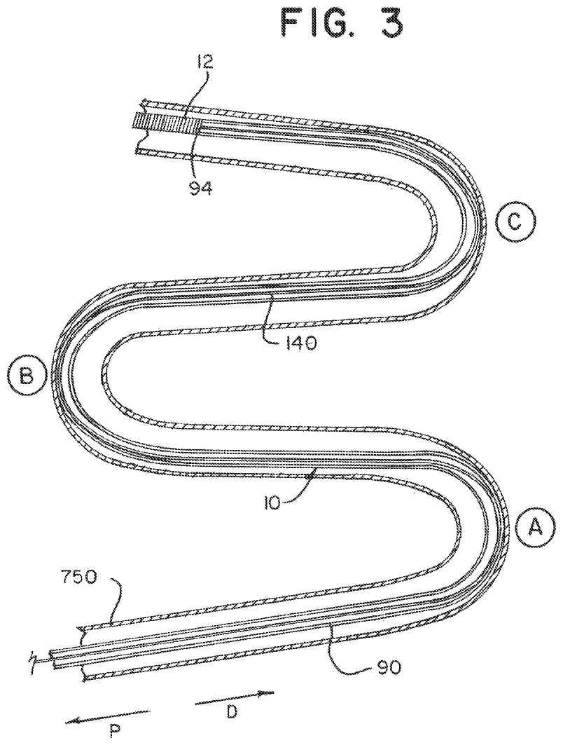

FIG. 3 illustrates positioning of an implant 12 such as an embolic coil suitable for aneurysm treatment, a guide catheter 750, and a delivery system 10 including an outer tubular body 90 and a pull wire 140 within tortuous vasculature (vasculature not illustrated). At bends A, B, and C, the outer tubular body 90 can extend to a sidewall of the guide catheter 750 on each outer curve of each bend, and likewise, the pull wire 140 can extend to a sidewall of the inner tubular body 190 (not shown) on each outer curve of each bend. Likewise, the inner tubular body 190 can extend to a sidewall of the outer tubular body 90 at bends A, B, C. During a procedure, the detachment system 10 including pull wire 140 can be fed into the guide catheter 750 in the distal direction D, first passing through bend A, then bend B, and then bend C. As the detachment system 10 navigates the bends, the pull wire 140 can be prevented from translating proximally with respect to the tubular body 90. The interference feature 210a, 210b ensures that the inner tubular body 190 cannot translate proximally with respect to outer tubular body 90 by holding the inner tubular body 190 in alignment with the outer tubular body 90 while the detachment system 10 navigates the tortuous vasculature of the patient to a treatment site.

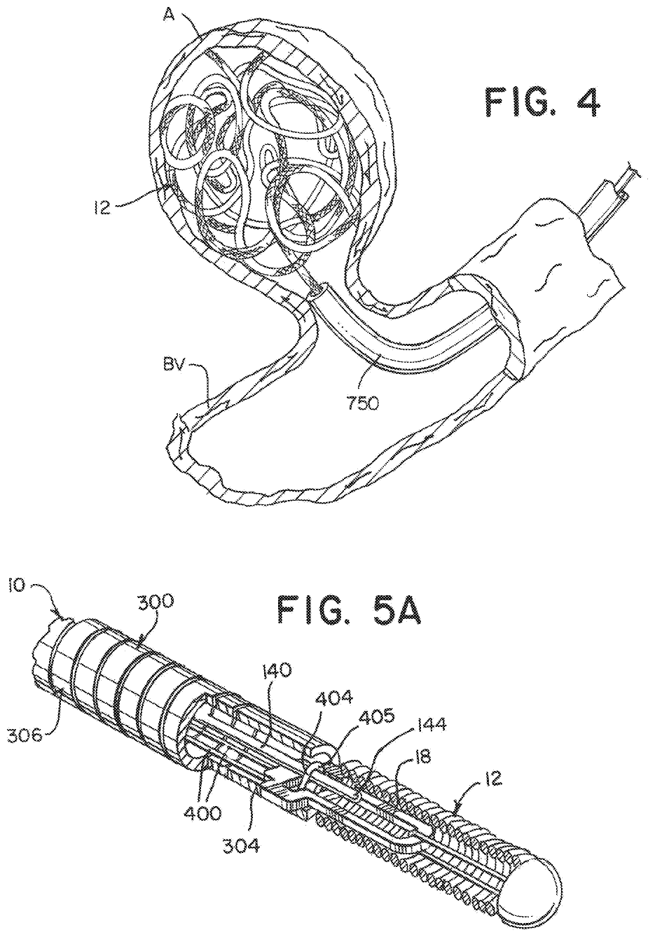

FIG. 4 is an illustration of embolic implant 12 being delivered through catheter 250 and positioned within an aneurysm A on a blood vessel BV. The implant can loop and bend with the aneurysm sac to form a thrombotic mass. The implant can loop back on themselves and/or loop next to other implants. As the aneurysm A becomes increasingly packed, overlapping portions of the implant 12 can press into each other.

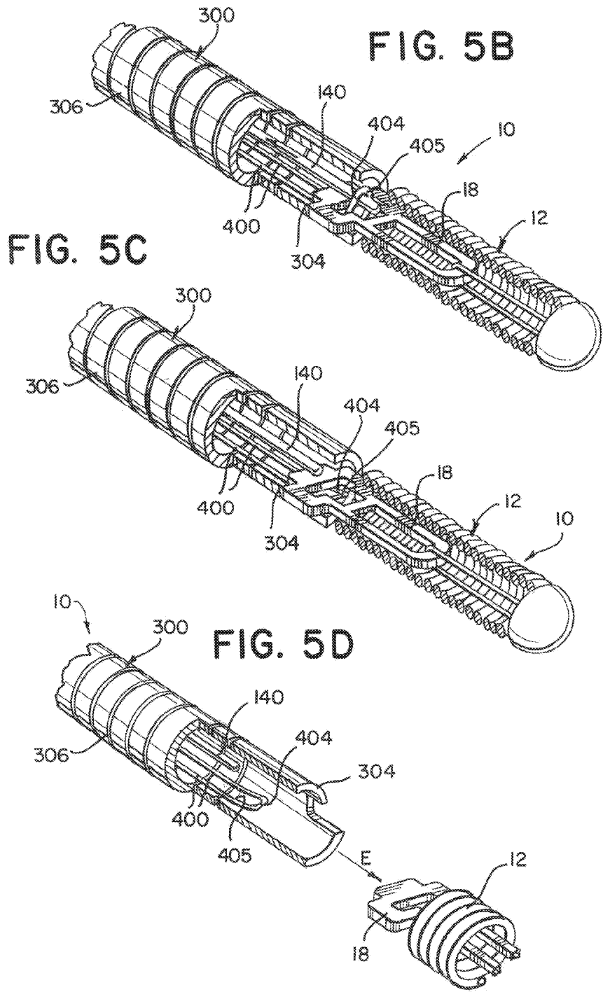

FIGS. 5A-5D illustrate a time sequence of steps for releasing an embolic implant 12 from a delivery system 10. The delivery system 10 can be configured such as illustrated in the previous figures and as otherwise described herein. FIG. 5A illustrates an engagement system including the loop wire 400 and pull wire 140 locked into a locking portion 18 of the medical device 12. The spiral cuts 306 of the compressible distal portion 300 can be compressed and the loop wire 400 opening 405 at a distal end 404 of the loop wire 400 can be placed through the locking portion 18. When the pull wire 140 is put through the opening 405 the medical device 12 is now secure. FIG. 5B illustrates the pull wire 140 being drawn proximally to begin the release sequence for the medical device 12. FIG. 5C illustrates the instant the distal end 144 of the pull wire exits the opening 405 and the pull wire 140 is pulled free of the loop wire 400. The distal end 404 of the loop wire 400 falls away and exits the locking portion 18. As can be seen, there is now nothing holding the medical device 12 to the detachment system 10. FIG. 5D illustrates the end of the release sequence. Here, the compressible portion 306 has extended/returned to its original shape and “sprung” forward. An elastic force E is imparted by the distal end 305 of the compressible distal portion 300 to the medical device 12 to “push” it away to ensure a clean separation and delivery of the medical device 12.

The compressible portion 306 can have a difference in length (distance of compression) when measured in the compressed configuration and the original, uncompressed configuration of about 0.5 mm to about 0.75 mm. Greater elastic force E can be achieved by using a greater distance of compression. The distance of compression can be determined by the sizing of the loop wire 400, the shape of the locking portion 18, and the shape of the distal end 304 of the compressible distal portion 300.

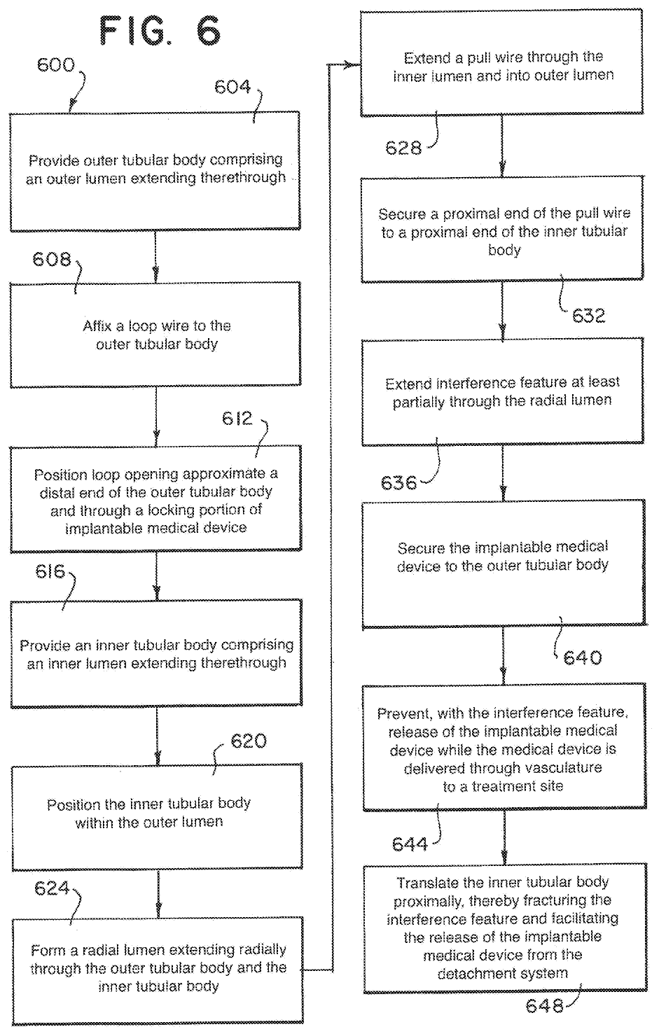

FIG. 6 is a flowchart of an example method of using the detachment system, according to aspects of the present invention. In block 604, the method can include providing an outer tubular body 90. The outer tubular body can include a first lumen extending therethrough along a longitudinal axis L-L. In optional block 608, the method can include affixing a loop wire to the outer tubular body 90. In optional block 612, the method can include positioning a loop opening 405 in the loop wire 400 approximate a distal end 94 of the outer tubular body 90. The loop opening 405 can be positioned through a locking portion 18 of the implantable medical device 12.

In block 616, the method can include providing an inner tubular body 190. Inner tubular body can include a second lumen extending therethrough along the longitudinal axis L-L. In block 620, the method can include positioning the inner tubular body 190 within the first lumen 708 of the outer tubular body 90. In block 624, the method can include forming a radial lumen 150 that extends radially through the outer tubular body 90 and the inner tubular body 190. The radial lumen can be disposed orthogonal to the longitudinal axis L-L.

In block 628, a pull wire can be extended through the second lumen 712 of the inner tubular body 190 and into the first lumen 708 of the outer tubular body 90. In block 632, a proximal end 142 of the pull wire 140 can be secured to a proximal end 192 of the inner tubular body 190. As discussed, the pull wire can be attached to the tubular body 190 using any method known to a person having skill in the pertinent art, for example by welding or gluing the proximal end 142 of the pull wire 140 to the proximal end 192 of the inner tubular body 190.

In block 636, an interference feature 210a, 210b can be extended at least partially through the radial lumen 150. The interference feature can extend through an outer wall 96 of the outer tubular body 90 and an inner wall 196 of the inner tubular body 90.

In block 640, an implantable medical device 12 can be secured to the outer tubular body such that proximal translation of the pull wire can release the implantable medical device from the outer tubular body 90.

In block 644, the interference feature can prevent release of the implantable medical device while the implantable medical device 12 is delivered through the vasculature to a treatment site. In block 648, the method can include translating the inner tubular body 190 proximally, thereby fracturing the interference feature 210a, 210b and facilitating release of the implantable medical device 12 from the detachment system 10.

According to some embodiments, the radial lumen can include an outer radial lumen 152 and an inner radial lumen 154. The outer radial lumen 152 can be in alignment with the inner radial lumen 154 as the implantable medical device 12 is delivered by the detachment system 10 to the target location of the body vessel. The inner radial lumen 154 can be configured to move out of alignment with respect to the outer radial lumen 152 to thereby fracture the interference feature 150 and facilitate release of the implantable medical device from the detachment system 10.

According to some embodiments, the interference feature 210a, 210b is configured to fracture in response to a force between approximately 50 gram-force and approximately 100 gram-force.

According to some embodiments, the method can include preventing proximal translation of the inner tubular body 190 with respect to the outer tubular body 90 while the implantable medical device 12 is delivered through vasculature to the treatment site.

According to some embodiments, securing the interference feature 210a, 210b to the outer wall 96 of the outer tubular body 90 using a method selected from welding the interference feature 210a, 210b to the outer wall 96, melting the interference feature 210a, 210b to the outer wall, knotting the interference feature 210a, 210b to the outer wall 96, and gluing the interference feature to the outer wall 96.

According to some embodiments, extending the interference feature 210a, 210b through the radial lumen 150 can further include extending a body 216 of the interference feature 210a, 210b through an entire length L1 of the radial lumen 150 and attaching first and second ends 212, 214 of the interference feature 210a, 210b to the outer wall 96 at respective ends of the radial lumen 150.

According to some embodiments, the body 216 of the interference feature 210a, 210b can be a monofilament suture. According to some embodiments, the pull wire 140 translates proximally with the inner tubular body 190 as a single unit.

The descriptions contained herein are examples of embodiments of the invention and are not intended in any way to limit the scope of the invention. As described herein, the invention contemplates many variations and modifications of the implantation system and associated methods, including alternative geometries of system components, alternative materials, additional or alternative method steps, etc. Modifications apparent to those skilled in the pertinent art are intended to be within the scope of the claims which follow.

1. A detachment system for delivering an implantable medical device to a target location of a body vessel, the detachment system comprising:
an outer tubular body comprising a first lumen extending therethrough along a longitudinal axis;
an inner tubular body comprising a second lumen extending therethrough along the longitudinal axis, the inner tubular body disposed within the first lumen of the outer tubular body;
a pull wire disposed at least partially within the second lumen of the inner tubular body and at least partially within the first lumen of the outer tubular body, the pull wire being configured to translate in a proximal direction in relation to the outer tubular body to release the implantable medical device from a distal end of the detachment system;
a radial lumen extending through the outer tubular body and the inner tubular body orthogonal to the longitudinal axis; and
an interference feature at least partially extending through the radial lumen,
wherein the interference feature is effective to inhibit premature detachment of the implantable medical device as the implantable medical device is delivered by the detachment system to the target location of the body vessel,
wherein the interference feature is configured to fracture in response to proximal translation of the inner tubular body, thereby facilitating release of the implantable medical device from the detachment system.

2. The detachment system of claim 1, wherein the interference feature comprises a mono filament suture.

3. The detachment system of claim 2, wherein the interference feature is constructed of polypropylene 8-0.

4. The detachment system of claim 1, wherein the interference feature is secured to an outer wall of the outer tubular body by a method selected from welding the interference feature to the outer wall, melting the interference feature to the outer wall, knotting the interference feature to the outer wall, and gluing the interference feature to the outer wall.

5. The detachment system of claim 1, wherein the interference feature prevents proximal translation of the inner tubular body with respect to the outer tubular body.

6. The detachment system of claim 1, wherein a proximal end of the pull wire is attached to a proximal end of the inner tubular body, the pull wire configured to translate with the inner tubular body as a single unit in relation to outer tubular body.

7. The detachment system of claim 1, the radial lumen comprising an outer radial lumen and an inner radial lumen,
the outer radial lumen in alignment with the inner radial lumen as the implantable medical device is delivered by the detachment system to the target location of the body vessel,
the inner radial lumen configured to move out of alignment with respect to the outer radial lumen, thereby fracturing the interference feature and facilitating release of the implantable medical device from the detachment system.

8. The detachment system of claim 1, wherein the interference feature extends through an entire length of the radial lumen and the interference feature is secured to an outer wall of the outer tubular body at each end of the radial lumen.

9. The detachment system of claim 1, wherein the interference feature is configured to fracture in response to a force between approximately 50 gram-force and approximately 100 gram-force.

10. The detachment system of claim 1, further comprising:
a loop wire comprising a loop opening positioned approximate a distal end of the outer tubular body,
wherein the loop wire and the pull wire are positioned to secure the implantable medical device to the detachment system.

11. The detachment system of claim 10, the outer tubular body further comprising a compressed distal portion held in compression by tension in the loop wire and configured to provide a force distally to the implantable medical device upon release of the implantable medical device from the distal end of the detachment system.

12. A method, comprising:
providing an outer tubular body of a detachment system, the outer tubular body comprising a first lumen extending therethrough along a longitudinal axis;
providing an inner tubular body of the detachment system, the inner tubular body comprising a second lumen extending therethrough along the longitudinal axis;
positioning the inner tubular body within the first lumen of the outer tubular body;
forming a radial lumen extending radially through the outer tubular body and the inner tubular body orthogonal to the longitudinal axis;
extending a pull wire through the second lumen of the inner tubular body and into the first lumen of the outer tubular body;
securing a proximal end of the pull wire to a proximal end of the inner tubular body;
extending an interference feature at least partially through the radial lumen such that the interference feature extends through an outer wall of the outer tubular body and an inner wall of the inner tubular body;
securing an implantable medical device to the outer tubular body such that proximal translation of the pull wire releases the implantable medical device from the outer tubular body;
preventing, with the interference feature, release of the implantable medical device while the implantable medical device is delivered through vasculature to a treatment site; and
translating the inner tubular body proximally, thereby fracturing the interference feature and facilitating the release of the implantable medical device from the detachment system.

13. The method of claim 12, wherein the radial lumen comprises an outer radial lumen and an inner radial lumen,
the outer radial lumen in alignment with the inner radial lumen as the implantable medical device is delivered by the detachment system to a target location of a body vessel, the inner radial lumen configured to move out of alignment with respect to the outer radial lumen, thereby fracturing the interference feature and facilitating release of the implantable medical device from the detachment system.

14. The method of claim 12, further comprising:
affixing a loop wire to the outer tubular body; and
positioning a loop opening in the loop wire approximate a distal end of the outer tubular body and through a locking portion of the implantable medical device.

15. The method of claim 13, wherein the interference feature is configured to fracture in response to a force between approximately 50 gram-force and approximately 100 gram-force.

16. The method of claim 12, further comprising:
preventing proximal translation of the inner tubular body with respect to the outer tubular body while the implantable medical device is delivered through vasculature to the treatment site.

17. The method of claim 12, further comprising securing the interference feature to the outer wall of the outer tubular body using a method selected from welding the interference feature to the outer wall, melting the interference feature to the outer wall, knotting the interference feature to the outer wall, and gluing the interference feature to the outer wall.

18. The method of claim 12, wherein extending the interference feature through the radial lumen further comprises:
extending a body of the interference feature through an entire length of the radial lumen; and
attaching first and second ends of the interference feature to the outer wall at respective ends of the radial lumen.

19. The method of claim 18, wherein the body of the interference feature comprises a mono filament suture.

20. The method of claim 13, wherein the pull wire translates proximally with the inner tubular body as a single unit.
